(19) United States
(12) Patent Application Publication
(10) Pub. No.: US 2026/0158816 A1
(43) Pub. Date: Jun. 11, 2026

(54) INK RECEPTACLE APPARATUS

(71) Applicant: Ember Catherine Seaman, Cary, NC (US)

(72) Inventor: Ember Catherine Seaman, Cary, NC (US)

(21) Appl. No.: 18/973,552

(22) Filed: Dec. 9, 2024

(51) Int. Cl. B43L 25/00; A61M 37/00

(52) U.S. Cl. CPC B43L 25/00; A61M 37/0076; A61M 2209/045

(57) ABSTRACT

An ink receptacle apparatus is provided. The ink receptacle apparatus can include a body portion continuously molded to an ink well at a first end, and continuously molded to a base at a second end. The ink well can be shaped to reduce ink waste by funneling ink to a central trough, or point. The ink well can be conical in shape, or alternatively can be shaped in a V-shaped valley configuration. The base can have a radius, or footprint, wider than the body thereby increasing stability of the apparatus.

### BACKGROUND OF THE INVENTION

The present invention relates to tools for use in body art designs, and, more particularly, to an ink receptacle apparatus configured to store tattoo ink.

Body art, such as tattooing, is a form of self-expression that has proliferated humanity for thousands of years. Tattooing is accomplished by a professional utilizing a tattoo gun and permanent inks of varying colors. A tip, or needle, of the tattoo gun is placed into an ink receptacle to receive ink and then placed on the skin of the person receiving the tattoo. Currently, ink receptacles are cylindrical with flat bottoms which poses problems for the tattoo artist when trying to receive ink. Specifically, the tip, or needle is a delicate apparatus which cannot be brought too close to the bottom of the ink receptacle resulting in the inability to utilize all ink in the receptacle. Additionally, the cylindrical shape can lead to instability in the tattooing environment when the tattoo artist is continually placing the tattoo gun into the ink receptacle. Finally, traditional ink receptacles for tattooing are uniform in design and are only differentiated by size, such as a metric measurement, which can lead to confusion and incorrect ordering.

As can be seen, there is a need for an improved ink receptacle apparatus for tattoo ink configured to reduce ink waste, provide improved overall stability, and eliminate confusion in sizing.

### SUMMARY OF THE INVENTION

In one aspect of the present invention, an ink receptacle apparatus configured to reduce ink waste is provided. The ink receptacle apparatus can include a substantially cylindrical body having a first end and a second end. An ink well can be continuously molded to the first end of the body and can be shaped to funnel ink to a central point, or trough. A base can be continuously molded to the second end of the body and can include a radius, or footprint, larger than that of the body thereby providing additional stability to the apparatus.

In another aspect of the present invention, the apparatus can have different sizes and shapes to accommodate differing ink amounts, and differing tools. The apparatus can be injection molded, 3-D printed, or manufactured utilizing other additive manufacturing capabilities. Furthermore, the apparatus can be formed of plastics, biodegradables, or compostables thereby reducing plastic waste.

These and other features, aspects and advantages of the present invention will become better understood with reference to the following drawings, description, and claims.

### BRIEF DESCRIPTION OF THE DRAWINGS

FIG. 1 is a front perspective view of embodiments of an ink receptacle apparatus, according to aspects of the present invention;

FIG. 2 is a front perspective view of a first embodiment of an ink receptacle apparatus, according to aspects of the present invention;

FIG. 3 is an elevation view of a group of a first embodiment of an ink receptacle apparatus showing varying sizes, according to aspects of the present invention;

FIG. 4 is an elevation view of a group of a second embodiment of an ink receptacle apparatus showing varying sizes, according to aspects of the present invention;

FIG. 5 is a top perspective view of a second embodiment of an ink receptacle apparatus, according to aspects of the present invention; and

FIG. 6 is a top perspective view of a first embodiment of an ink receptacle apparatus, according to aspects of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Tattoo inks are becoming more expensive, and current ink receptacles utilized by tattoo artists waste ink due to their flat bottom cylindrical shape. This is primarily due to the actuation of the tip, or needle of the tattoo gun, which if brought into contact with the ink receptacle could cause damage to the receptable, contamination of the tip with plastic debris, and/or dulling the tip. Additionally, these receptacles are susceptible to tipping, and spilling of costly ink, due to the continued introduction of the tattoo gun into the ink receptacle.

Broadly, one embodiment of the present invention is an ink receptacle apparatus configured to prevent ink waste due to receptacle shaping and improve stability in ink receptacles. The present invention includes a cylindrical body continuously formed to an internal ink well and a base having a wider footprint than the body to aid in stability. The ink well of the present invention can have a shape configured to save ink by funneling ink to a concentrated area. In differing embodiments of the present invention, the ink well can have different shapes, such as a valley, or V-shape, and/or a cone shape. The present invention can be manufactured from plastics, biodegradables, or compostables utilizing one or more of: injection molding, 3-D printing, or other additive manufacturing.

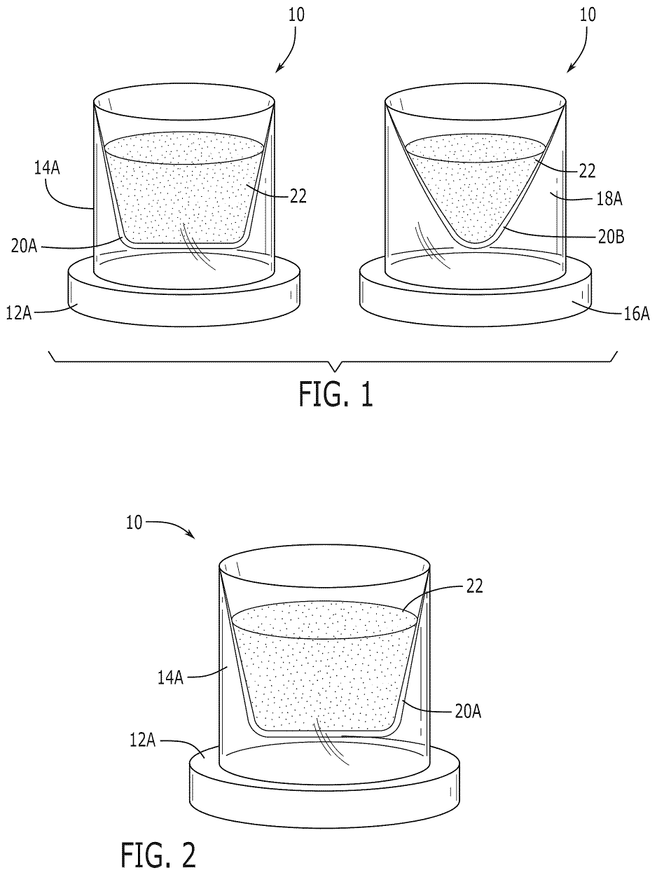

Referring to FIGS. 1-6, aspects of embodiments of an ink receptacle apparatus are disclosed. FIG. 1 illustrates perspective views of a first embodiment 14A and a second embodiment 18A of an ink receptacle apparatus 10. In embodiments, ink receptacle apparatus 10 can have a substantially cylindrical body forming an outer wall of ink receptacle apparatus 10. In embodiments, the body of apparatus 10 can be continuously formed to an ink well 20A and 20B configured to hold ink 22. In the first embodiment, ink well 20A can be shaped as a V-shaped conical valley. Ink well 20A can have a substantially circular, or oval, base portion as the interface between the body of apparatus 10 and the ink well 20A, converging to a V-shaped ridge at its trough. In the second embodiment, ink well 20B can be shaped in a substantially conical configuration. In embodiments, base 12A and base 16A can be substantially cylindrical in shape and can have a wider footprint, or radius, than that of the body of apparatus 10. Advantageously, the shape of ink wells 20A and 20B reduce ink waste by funneling ink to provide more ink depth across the volume of the well. Additionally, bases 12A and 16A can provide improved stability to apparatus 10 preventing accidental spillage of ink and further reducing waste.

Referring to the second embodiment 18A of an ink receptacle apparatus 10 in FIG. 1. The second embodiment 18A can have a substantially cylindrical body that forms an outer wall of apparatus 10, but is not so limited as shapes such as polygonal prisms are equally contemplated. Additionally, The substantially cylindrical body can take any elliptical shape, such as an oval, and or circle. The body can be continuously molded to an ink well 20B forming an opening at a first base of the substantially cylindrical body. In embodiments the opening can be circular, ovular, or otherwise shaped to accept a tattooing apparatus. In the second embodiment, ink well 20B can be shaped as a conical in shape having a rounded, hemispherical, or semispherical, apex or trough. Additionally, the second embodiment 18A can include a base 16A which can be substantially cylindrical in shape, but is not so limited as shapes such as polygonal prisms are equally contemplated. In embodiments, base 16A can have a radius, or footprint, larger than the body of apparatus 10 to provide additional stability.

FIG. 2 illustrates a perspective view of a first embodiment 14A of an ink receptacle apparatus 10. The first embodiment 14A can have a substantially cylindrical body that forms an outer wall of apparatus 10, but is not so limited as shapes such as polygonal prisms are equally contemplated. Additionally, The substantially cylindrical body can take any elliptical shape, such as an oval, and or circle. The body can be continuously molded to an ink well 20A forming an opening at a first base of the substantially cylindrical body. In embodiments the opening can be circular, ovular, or otherwise shaped to accept a tattooing apparatus. In embodiments, the opening can be ovular in shape with the major axis of the oval parallel to the trough, or valley of inkwell 20A. In the first embodiment, ink well 20A can be shaped as a V-shaped cone with a valley portion. For example, ink well 20A can be shaped such that a first cross section taken parallel to the valley portion would show a trapezoidal shape, while a second cross section taken perpendicular to the valley portion would show a V-shape (as illustrated in FIG. 4). Additionally, the first embodiment 14A can include a base 12A which can be substantially cylindrical in shape, but is not so limited as shapes such as polygonal prisms are equally contemplated. In embodiments, base 12A can have a radius, or footprint, larger than the body of apparatus 10 to provide additional stability.

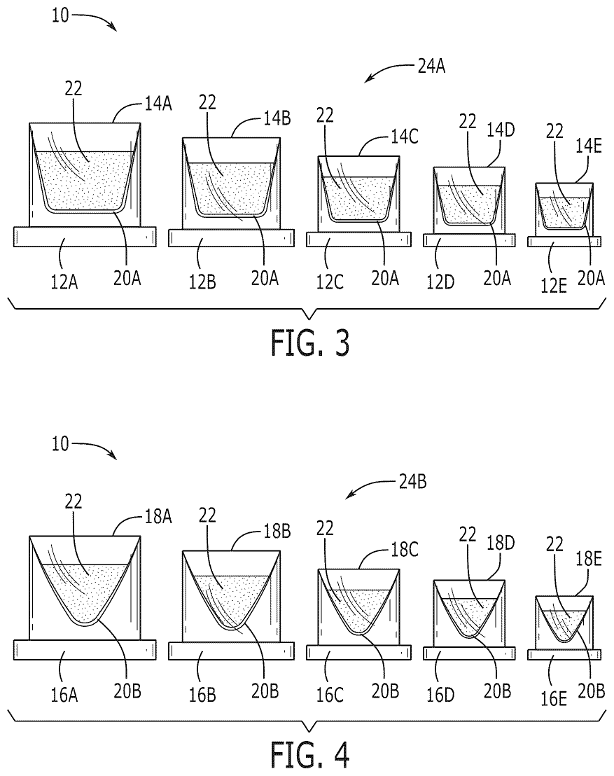

FIGS. 3-4 illustrate perspective views of the first embodiment 14A-E and the second embodiment 18A-E. In each embodiment, the size of the body and the size of base 12A-E and 16A-E can be varied to support differing volumes of ink 22. In each embodiment, base 12A-E and 16A-E can be emphasized to indicate a size, or volume of each of the respective embodiments. For example, base 12A-E and 16A-E can be color coded to indicate a size, or volume of each of the respective embodiments, where a respective color indicates a fixed size. Advantageously, color coding bases 12A-E and 16A-E in this manner eliminates confusion as to sizing when searching, browsing, and/or purchasing apparatus 10.

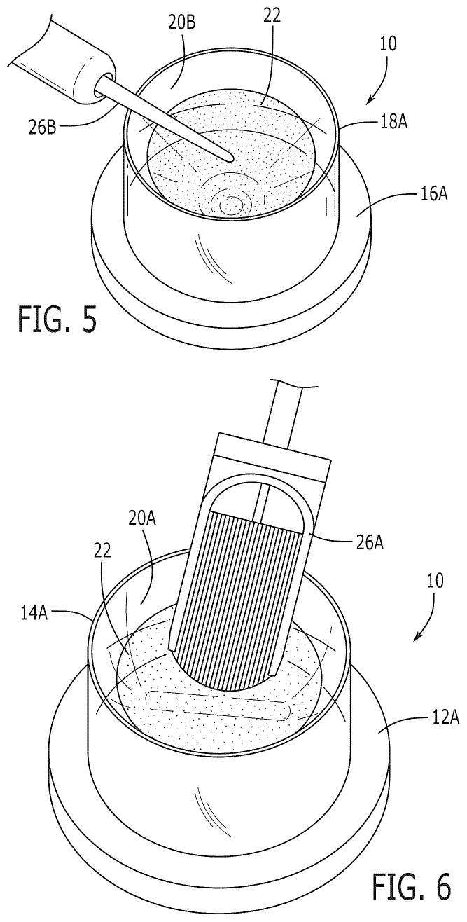

FIG. 5 illustrates a top perspective view of the second embodiment 18A of an ink receptacle apparatus 10 show in-use. To utilize the second embodiment 18A a tattoo artist, or user, can introduce a liner needle 26B into ink well 20B thereby retrieving ink 22. In embodiments, the shape of ink well 20B funnels ink 22 to a central location in apparatus 10, thereby providing a greater concentration of ink 22 in a smaller volume when compared to prior art flat bottom ink receptacles. Advantageously, the shape of ink well 20B allows for the retrieval of more ink than prior art ink wells thereby reducing ink waste.

FIG. 6 illustrates a top perspective view of the first embodiment 14A of an ink receptacle apparatus 10 show in-use. To utilize the second embodiment 14A a tattoo artist, or user, can introduce a magnum needle 26A into ink well 20A thereby retrieving ink 22. In embodiments, the shape of ink well 20A funnels ink 22 to a central ridge in apparatus 10, thereby providing a greater concentration of ink 22 in a smaller volume when compared to prior art flat bottom ink receptacles. The shape of ink well 20A, and specifically the ridge, allows for the proper introduction of magnum needle 26A without adding additional space for ink 22 to disperse. Advantageously, the shape of ink well 20A reduced ink waste.

It should be understood, of course, that the foregoing relates to exemplary embodiments of the invention and that modifications may be made without departing from the spirit and scope of the invention as set forth in the following claims.

What is claimed is:

1. An ink receptacle apparatus comprising:
a substantially cylindrical body having a first end and a second end;
an ink well continuously molded to the first end; and
a base continuously molded to the second end.

2. The ink receptacle apparatus of claim 1, wherein the ink well is substantially conical in shape.

3. The ink receptacle apparatus of claim 1, where a shape of the ink well comprises:
a trough portion including a longitudinal axis, wherein the shape of the well is V-shaped when viewed from a cross-section perpendicular to the longitudinal axis, and trapezoidal shaped when view from a cross-section parallel to the longitudinal axis.

4. The ink receptacle apparatus of claim 1, wherein the base is substantially cylindrical in shape.

5. The ink receptacle apparatus of claim 4, wherein a radius of the base is larger than a radius of the body.
